(19) United States
(12) Patent Application Publication
(10) Pub. No.: US 2004/0091441 A1
(43) Pub. Date: May 13, 2004

(54) Cosmetic preparations for shaving using a shaving device

(75) Inventors: Kerstin Heike, Hamburg (DE); Waltraud Kaden, Halstenbek (DE); Sven Gohla, Hamburg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignees: Beiersdorf AG; Koninklijke Philips Electronics

(21) Appl. No.: 10/443,585

(22) Filed: May 22, 2003

Related U.S. Application Data
(63) Continuation of application No. PCT/EP01/13512, filed on Nov. 21, 2001.

(30) Foreign Application Priority Data
Nov. 22, 2000 (DE) 100 57 925.6

(51) Int. Cl. A61K 7/06; A61K 7/11; A61K 7/075; A61K 7/08

(52) U.S. Cl. 424/70.13; 424/70.24

(57) ABSTRACT

Cosmetic preparations, in particular shaving gels, which are suitable for shaving using a mechanical shaving device, in particular an electric shaving device, characterized by a content of

(1) one or more N-acyl sarcosinates,

(2) one or more polysaccharides,

(3) one or more substances which form a gel structure in water, which are not polysaccharides and which advantageously have interface-active properties,

(4) one or more solubility promoters, preferably chosen from the group of polyethoxylated saturated fat or oil components, and

(5) water.

### CROSS-REFERENCE TO RELATED APPLICATIONS

This is a continuation application of PCT/EP01/13512, filed Nov. 21, 2001, which is incorporated herein by reference in its entirety, and also claims the benefit of German Priority Application No. 100 57 925.6, filed Nov. 22, 2000.

### Field of the Invention

The invention relates to cosmetic preparations, in particular shaving gels, which are suitable for shaving using a mechanical shaving device, in particular an electric shaving device. In particular, the invention relates to those shaving gels which are intended to be applied to the skin before or during electric shaving.

### Background of the Invention

The growth of beard hair is triggered in the adolescent man by the increased formation of male hormones during puberty. Hormonal imbalances in women can likewise lead to a form of beard growth, which despite being thoroughly unpleasant, is usually significantly less marked in its severity than that of male beard growth.

Shaving can be motivated (also made taboo) by various constraints, e.g. of a religious or cultural nature; in the simplest case, beard growth is simply undesired for cosmetic reasons for the person concerned.

Shaving is either carried out dry or wet. Whereas in the case of wet shaving chemical auxiliaries are essential, these are also at least advisable in the case of dry shaving in order to effect the closest possible shave, i.e. to cut the beard hair as close as possible to the surface of the skin.

For the purposes of the present disclosure, to simplify the language usage, the term “electric shaving” and synonyms are always used when talking about mechanical shaving devices, in particular electric shaving devices, since these, as well as wet shaving blades, represent the only significant shaving devices in practice.

For electric shaving, which is also referred to as dry shaving, the beard hair must be stiff, in contrast to “wet shaving”, in order that it can be gripped by the shaving heads of the electric shavers. In the past this meant it also had to be dry and therefore brittle. For this purpose, “preshaves” (also: “electric preshaves”) were applied, mostly highly alcoholic preparations (typically around 80% by weight of ethanol), through the evaporation of which moisture from the beard hair was also entrained.

A disadvantage of the known shaving preparations is that they produce only inadequate lubricity between the shaving head and the surface of the skin. This is particularly the case when highly alcoholic preparations are applied which leave behind a sebum layer of low lubricity on the surface of the skin.

In addition, it is a shortcoming of the prior art that using the conventional preparations a thorough shaving can be effected only to a limited degree, that the electrical contacts of the electric shaver can be impaired, damaged or even destroyed by constituents of the formulations, that other metal parts are also subjected to corrosion and/or that the beard stubble which forms in the hair-collecting chamber of the electric shaver, mixed with the shaving preparation, leads to mixtures which are difficult to remove even with water. The latter additionally harbor the risk of being nutrient media for microorganisms.

WO98/08659 describes a shaving system which comprises an electric shaver and a container. The shaver comprises a shaving head and a hair-collecting chamber which can accommodate the container. The container comprises a reservoir which is filled with a shaving preparation, where the shaving preparation can be applied to the skin through appropriate devices in the shaving head.

Shaving preparations in liquid form for such purposes are known per se. Usually, they are liquid or semi-liquid to pasty aqueous, gel-like preparations or preparations in the form of emulsions.

WO98/47474 describes, for example, a shaving liquid for a shaving system which comprises water as the main constituent, and also customary additives and a surface-active agent, where the shaving liquid is an emulsion of at least one liquid hydrocarbon in water and the surface-active agent is a C₆-C₁₈-alkyl glucoside.

A further disadvantage is that the preparations develop lather during application or during shaving since this lather interferes with shaving with an electric shaving device to a considerable degree.

A further disadvantage is also that shaving, including electric shaving, irritates the facial skin to a greater or lesser degree. Shaving preparations should thus for their part not add to the irritancy potential to a noteworthy extent.

Customary cosmetic and dermatological preparation forms which have become ever more widespread in recent times are gels.

In the technical sense, gels are understood as meaning: relatively dimensionally stable, readily deformable disperse systems of at least two components, which usually consist of an (in most cases solid) colloidally dispersed substance of long-chain molecule groups (e.g. gelatin, silica, polysaccharides) as structure former and a liquid dispersant (e.g. water). The colloidally dispersed substance is often referred to as a thickener or gelling agent. It forms a spatial network within the dispersant, where individual colloidal particles may be joined together with greater or lesser strength via electrostatic interaction. The dispersant which surrounds the network is characterized by electrostatic affinity to the gelling agent, i.e. a predominantly polar (in particular: hydrophilic) gelling agent preferably gels a polar dispersant (in particular: water), whereas a pre-dominantly nonpolar agent preferably gels nonpolar dispersants.

Strong electrostatic interactions which are realized, for example, in hydrogen bridge bonds between gelling agent and dispersant, but also between dispersant molecules with one another, can lead to considerable crosslinking also of the dispersant. Hydrogels can consist of virtually 100% of water (in addition, for example, to about 0.2-1.0% of a gelling agent) and have an entirely solid consistency. The water content is present here in ice-like structural elements, meaning that gels therefore do justice to the origin of their name [from Latin “gelatum” =“frozen” via the alchemistic term “gelatina” (16^{th} century) for the modern term “gelatin”].

In cosmetics and pharmaceutical technology, lipogels and oleogels (of waxes, fats and fatty oils), and also carbogels (of paraffin or petrolatum) are also common. In practice, a distinction is made between oleogels, which are virtually free from water, and hydrogels which are virtually free from fat. In most cases, gels are transparent. In cosmetic or pharmaceutical technology, gels are usually characterized by a semisolid, often flowable consistency.

In addition, so-called surfactant gels are customary preparations of the prior art. This is understood as meaning systems which, as well as water, have a high concentration of emulsifiers, typically more than about 25% by weight, based on the overall composition. If oil components are solubilized in these surfactant gels, microemulsion gels are obtained which are also referred to as “ringing gels”. By adding nonionic emulsifiers, for example alkyl polyglycosides, it is possible to obtain cosmetically more elegant microemulsion gels.

Although gels are commonly regarded as being particularly skin-friendly, they also usually typically have a relatively high content of substances which give rise to at least some of the shortcomings described at the outset to a greater or lesser degree.

### SUMMARY OF THE INVENTION

It has now been found that cosmetic preparations, in particular shaving gels, which are suitable for shaving using a mechanical shaving device, in particular an electric shaving device, characterized by a content of

(1) one or more N-acyl sarcosinates,

(2) one or more polysaccharides,

(3) one or more substances which form a gel structure in water, which are not polysaccharides and which advantageously have interface-active properties,

(4) one or more solubility promoters, preferably chosen from the group of polyethoxylated saturated fat or oil components, and

(5) water

overcome the disadvantages of the prior art.

The preparations according to the invention have an extraordinarily low skin irritancy potential. Surprisingly, they produce high lubricity between the shaving head and the surface of the skin and ensure improved shaving without the metal parts of an electric shaver to be used running the risk of being subjected to a corrosive influence. In the case of the use according to the invention, the shaving area of the electric shaver can be cleaned easily with water, even without further cleaning additives, without troublesome residues being left behind. The use of the preparation according to the invention for shaving using shaving systems as in or analogous to WO98/08659 is particularly advantageous.

### DETAILED DESCRIPTION OF THE INVENTION

N-acyl sarcosinates are surfactants which can, in principle, be regarded as being “interrupted soaps” because the only difference between a soap (fatty acid) and the sar-cosinate is that the fatty acid is interrupted by the insertion of a methylamido group:

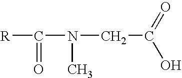

R is preferably lauroyl, cocoyl, myristoyl, oleyl, stearyl or a mixture of these radicals. Particular preference is given to sodium lauroyl sarcosinate (CAS No. 137-16-6). It is supplied as a 30% strength aqueous solution (for example under the trade names SARKOSYL®) NL-30, MAPROSYL® 30 and ROKOSYL® NL 30).

According to the invention, the preparations advantageously comprise 0.01 to 10% by weight, particularly advantageously 0.01 to 5% by weight, very particularly preferably 0.5 to 3% by weight, of one or more N-acyl sarcosinates, in each case based on the total weight of the preparations.

It is advantageous to use polysaccharides which are water-soluble and/or swellable in water and/or gellable using water.

An advantageous polysaccharide for the purposes of the present invention is, for example, carrageen, a gel-forming extract with a similar structure to agar, of North Atlantic red algae which belong to the Florideae (Chondrus crispus and Gigartina stellata).

The term carrageen is often used for the dried algae product and carrageenan for the extract thereof. The carrageen precipitated from the hot-water extract of the algae is a colorless to sand-colored powder with a molecular weight range from 100 000-800 000 and a sulfate content of about 25%. Carrageen, which is very readily soluble in warm water, forms a thixotropic gel upon cooling, even if the water content is 95-98%. The rigidity of the gel is effected by the double helix structure of the carrageen. In the case of carrageen, a distinction is made between three main constituents: the gel-forming κ fraction consists of D-galactose 4-sulfate and 3,6-anhydro-α-D-galactose, which have alternate glycoside bonds in the 1,3 and 1,4 position (by contrast, agar contains 3,6-anhydro-α-L-galactose). The nongelling λ fraction is composed of 1,3-glycosidically linked D-galactose 2-sulfate and 1,4-bonded D-galactose-2,6-disulfate radicals, and is readily soluble in cold water. ι-Carrageenan, composed of D-galactose 4-sulfate in 1,3 bond and 3,6-anhydro-α-D-galactose 2-sulfate in 1,4 bond is both water-soluble and also gel-forming. Further carrageen types are likewise referred to with Greek letters: α, β, γ, μ, ν, ξ, π, ω, χ. The nature of the cations present (K⁺, NH₄⁺, Na⁺, Mg²+, Ca²+) also influences the solubility of the carrageens.

It is also of particular advantage to use hyaluronic acid, chitosan and also other polysaccharides which are water-soluble and/or water-swellable and/or gellable using water.

Hyaluronic acid is characterized by the structure

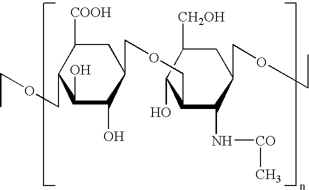

If hyaluronic acid is the or one of the polysaccharides used, it is advantageous to choose those with molecular weights between 30 000 and 8 000 000, in particular those with molecular weights between 500 000 and 1 500 000.

Chitosan is characterized by the following structural formula:

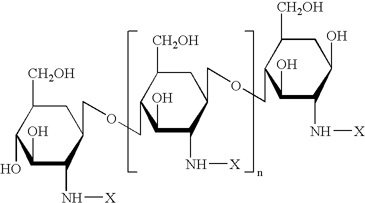

Here, n assumes values up to about 2000, and X is either the acetyl radical or hydrogen. Chitosan forms by deacetylation and partial depolymerization (hydrolysis) of chitin, which is characterized by the structural formula

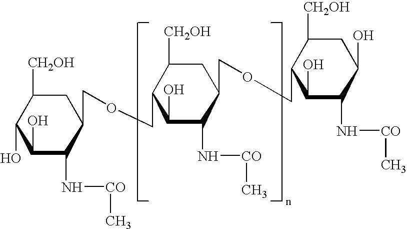

Chitin is an essential constituent of the ectoskeleton [″o χιτων=Greek: integument] of arthropods (e.g. insects, crabs, spiders) and is also found in supporting tissues of other organisms (e.g. mollusks, algae, fungi). Chitosan is a raw material known in hair care. It is suitable, more so than the chitin on which it is based, as a thickener or stabilizer and improves the adhesion and water resistance of polymeric films. Representative of a large number of references of the prior art: H. P. Fiedler, “Lexikon der Hilfsstoffe fur Pharmazie, Kosmetik und angrenzende Gebiete” [Lexikon of auxiliaries for pharmacy, cosmetics and related fields], third edition 1989, Editio Cantor, Aulendorf, p. 293, keyword “chitosan”.

According to the invention, preference is given to chitosans with a degree of deacetylation of >60%, in particular >80%. Among these, particular preference is given to those whose 1% strength aqueous solution has a viscosity of 4500-5500 mPas (Brookfield, Spindel 5, 10 rpm), in particular 5000 mpas.

If chitosan is one of the polysaccharides used, it is advantageous to choose one with molecular weights between 3000 and 2 000 000, in particular one with molecular weights between 10 000 and 500 000.

A preferred polysaccharide is xanthan gum (CAS No. 11138-66-2), which is an anionic heteropolysaccharide which is generally formed by fermentation from maize sugar and is isolated as the potassium salt. It is produced by Xanthomonas campestris and some other species under aerobic conditions and has a molecular weight of from 2×10⁶ to 24×10⁶. Xanthan is formed from a chain having β-1,4-bonded glucose (cellulose) with side chains. The structure of the subgroups consists of glucose, mannose, glucuronic acid, acetate and pyruvate. Xanthan is the name for the first microbial anionic heteropolysaccharide. It is produced from Xanthomonas campestris and some other species under aerobic conditions and has a molecular weight of 2-15 10⁶. Xanthan is formed from a chain with β-1,4-bonded glucose (cellulose) with side chains. The structure of the subgroups consists of glucose, mannose, glucuronic acid, acetate and pyruvate. The number of pyruvate units determines the viscosity of the xanthan. Xanthan is produced in two-day batch cultures with a yield of 70-90%, based on the carbohydrate used. Yields of 25-30 g/l are achieved. Work-up takes place by killing the culture by precipitation with, for example, 2-propanol. Xanthan is then dried and ground. Xanthan is characterized by the structure

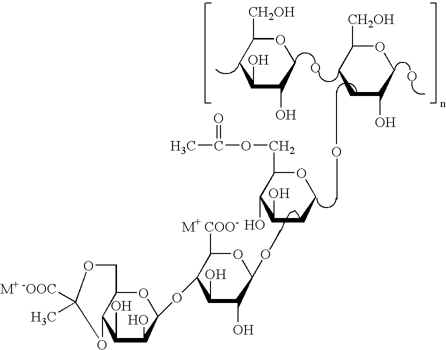

where M⁺ can be Na⁺, K⁺ or half a Ca²⁺ equivalent.

According to the invention, the preparations advantageously comprise 0.01 to 10% by weight, particularly advantageously 0.01 to 5% by weight, very particularly preferably 0.5 to 3% by weight, or one or more polysaccharides, in each case based on the total weight of the preparations.

The substance or substances which form a gel structure in water, which do not represent polysaccharides and which advantageously have interface-active properties is or are preferably chosen from the group of polyacrylates. Polyacrylates advantageous according to the invention are acrylate-alkyl acrylate copolymers, in particular those chosen from the group of so-called carbomers or carbopols (CARBOPOL®is a registered trademark of B.F. Goodrich Company). In particular, the acrylate-alkyl acrylate copolymers advantageous according to the invention are characterized by the following structure:

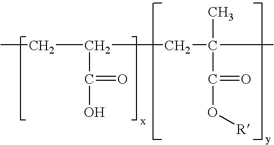

In this structure, R″ is a long-chain alkyl radical and x and y are numbers which symbolize the respective stoichiometric proportion of each of the comonomers.

Also advantageous are copolymers of C₁₀₋₃₀-alkyl acrylates and one or more monomers of acrylic acid, of methacrylic acid or esters thereof which are crosslinked with an allyl ether of sucrose or an allyl ether of pentaerythritol.

According to the invention, the following polyacrylate types are particularly advantageous:

| | |
|---|---|
| Carbomer 907 | (Mol. wt. 450 000), |
| Carbomer 910 | (Mol. wt. 750 000), |
| Carbomer 941 and | (Mol. wt. 1 250 000), |
| Carbomer 951 | |
| Carbomer 934, | (Mol. wt. 3 000 000), |
| Carbomer 940 and | |
| Carbomer 954 | |
| Carbomer 940 | (Mol. wt. 4 000 000), |
| Carbomer 980, | (Mol. wt. 4 000 000), |
| Carbomer 981 | (Mol. wt. 1 250 000), |
| Carbomer 984 | (Mol. wt. 3 000 000), |

(Carbomer 980, 981 and 984 are acrylic acid polymers which are polymerized in a mixture of ethyl acetate and cyclohexane)

| | |
|---|---|
| Carbomer 974 P | (Mol. wt. 3 000 000), |
| Carbomer 1342 | (Mol. wt. 1 300 000). |

Carbomer 1342 has a similar composition to the PEMULEN® products. PEMULEN® is the trade name for a copolymer of C₁₀₋₃₀-alkyl acrylates and one or more monomers of acrylic acid, of methacrylic acid or esters thereof which are crosslinked with an allyl ether of sucrose or an allyl ether of pentaerythritol (CTFA name: Acrylates/C₁₀₋₃₀ Alkyl Acrylate Crosspolymer). The PEMULEN® products are high molecular weight polymers with a high hydrophilic fraction and simultaneously with a low content of lipophilic moieties.

Carbomer 1342 is the preferred substance which forms a gel structure in water according to the invention.

According to the invention, the preparations advantageously comprise 0.01 to 10% by weight, particularly advantageously 0.01 to 5% by weight, very particularly preferably 0.5 to 3% by weight, of one or more substances which form a gel structure in water, which do not represent polysaccharides, in each case based on the total weight of the preparations.

The solubility promoter(s) is/are advantageously chosen from the group of polyethoxylated saturated fat or oil components, in particular from the group PEG-X hydrogenated castor oil, where X can advantageously assume the numbers 40, 100 or 200.

According to the invention, the preparations advantageously comprise 0.01 to 10% by weight, particularly advantageously 0.01 to 5% by weight, very particularly preferably 0.5 to 3% by weight, or one or more solubility promoters, in each case based on the total weight of the preparations.

The water content of the preparations according to the invention is generally between 70 and 98% by weight, based on the total weight of the preparations, in particular between 85 and 95% by weight.

It is advantageous according to the invention to add further thickeners to the preparations, in particular, for example, methylcelluloses, which is the term used for the methyl ethers of cellulose. They are characterized by the following structural formula

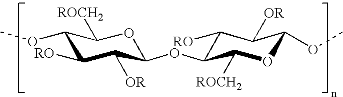

in which R may be a hydrogen or a methyl group.

Particularly advantageous for the process of the present invention are the cellulose mixed ethers, which are generally likewise referred to as methylcelluloses, which contain, in addition to a dominating content of methyl groups, additionally 2-hydroxyethyl, 2-hydroxypropyl or 2-hydroxybutyl groups. Particular preference is given to (hydroxypropyl)methylcelluloses, for example those available under the trade name METHOCEL E4M from Dow Chemical.

Also advantageous according to the invention is sodium carboxymethylcellulose, the sodium salt of the glycolic ether of cellulose, for which R in structural formula I can be a hydrogen and/or CH₂-COONa. Particular preference is given to the sodium carboxymethylcellulose available under the trade name NATROSOL Plus 330 CS from Aqualon and referred to as cellulose gum.

It may in some instances be advantageous according to the invention to add one or more further surfactants to the preparations apart from the sarcosinates. Surfactants are amphiphilic substances which are able to dissolve organic nonpolar substances in water. As a result of their specific molecular structure having at least one hydrophilic and one hydrophobic molecular moiety, they are able to reduce the surface tension of the water, wet the skin, facilitate the removal and release of dirt, facilitate rinsing and—if desired—control foaming.

The hydrophilic moieties of a surfactant molecule are mostly polar functional groups, for example —COO⁻, —OSO₃²⁻, —SO₃₋, while the hydrophobic moieties are usually nonpolar hydrocarbon radicals. Surfactants are generally classified according to the type and charge of the hydrophilic molecular moiety. In this connection, it is possible to differentiate between four groups:

anionic surfactants,

cationic surfactants,

amphoteric surfactants and

nonionic surfactants.

Anionic surfactants usually have, as functional groups, carboxylate, sulfate or sulfonate groups. In aqueous solution, they form negatively charged organic ions in acidic or neutral media. Cationic surfactants are characterized virtually exclusively by the presence of a quaternary ammonium group. In aqueous solution they form positively charged organic ions in acidic or neutral media. Amphoteric surfactants contain both anionic and cationic groups and accordingly, in aqueous solution, exhibit the behavior of anionic or cationic surfactants depending on the pH. In strongly acidic media they have a positive charge, and in alkaline media a negative charge. By contrast, in the neutral pH range, they are zwitterionic, as the example below is intended to illustrate:

RNH₂⁺CH₂CH₂COOH X⁻(at pH=2) X⁻=any anion, e.g. Cl⁻

RNH₂⁺CH₂CH₂COO⁻(at pH=7)

RNHCH₂CH₂COO⁻B⁺(at pH=12) B⁺=any cation, e.g. Na⁺

Polyether chains are typical of nonionic surfactants. Nonionic surfactants do not form ions in an aqueous medium.

A. Anionic Surfactants

Anionic surfactants which can be used advantageously are: acylamino acids (and salts thereof), such as

1. acyl glutamates, for example sodium acyl glutamate, di-TEA palmitoyl aspartate and sodium caprylic/capric glutamate,

2. acylpeptides, for example palmitoyl-hydrolyzed milk protein, sodium cocoyl-hydrolyzed soy protein and sodium/potassium cocoyl-hydrolyzed collagen,

3. taurates, for example sodium lauroyl taurate and sodium methylcocoyl taurate,

4. acyl lactylates, lauroyl lactylate, caproyl lactylate

5. alaninates,

carboxylic acids and derivatives, such as

1. carboxylic acids, for example lauric acid, aluminum stearate, magnesium alkanolate and zinc undecylenate,

2. ester carboxylic acids, for example calcium stearoyl lactylate, laureth-6 citrate and sodium PEG-4 lauramide carboxylate,

3. ether carboxylic acids, for example sodium laureth-13 carboxylate and sodium PEG-6 cocamide carboxylate,

phosphoric esters and salts, such as, for example, DEA-oleth-10 phosphate and dilaureth-4 phosphate,

sulfonic acids and salts, such as

1. acyl isethionates, e.g. sodium/ammonium cocoyl isethionate,

2. alkylarylsulfonates,

3. alkylsulfonates, for example sodium cocomonoglyceride sulfate, sodium C₁₂₋₁₄-olefinsulfonate, sodium lauryl sulfoacetate and magnesium PEG-3 cocamide sulfate,

4. sulfosuccinates, for example dioctyl sodium sulfosuccinate, disodium laureth sulfosuccinate, disodium lauryl sulfosuccinate and disodium undecyleneamido MEA sulfosuccinate and

sulfuric esters, such as

1. alkyl ether sulfate, for example sodium, ammonium, magnesium, MIPA, TIPA laureth sulfate, sodium myreth sulfate and sodium C₁₂₋₁₃ pareth sulfate,

2. alkyl sulfates, for example sodium, ammonium and TEA lauryl sulfate.

B. Cationic Surfactants

Cationic surfactants which can be used advantageously are

1. alkylamines,

2. alkylimidazoles,

3. ethoxylated amines and

4. quaternary surfactants,

5. ester quats.

Quaternary surfactants contain at least one N atom which is covalently bonded to 4 alkyl and/or aryl groups. Irrespective of the pH, this leads to a positive charge. Advantageous quaternary surfactants are alkylbetaine, alkylamidopropylbetaine and alkylamidopropylhydroxysulfaine. For the purposes of the present invention, cationic surfactants can also preferably be chosen from the group of quaternary ammonium compounds, in particular benzyltrialkylammonium chlorides or bromides, such as, for example, benzyldimethylstearylammonium chloride, and also alkyltrialkylammonium salts, for example cetyltrimethylammonium chloride or bromide, alkyldimethylhydroxyethylammonium chlorides or bromides, dialkyldimethylammonium chlorides or bromides, alkylamidoethyltrimethylammonium ether sulfates, alkylpyridinium salts, for example lauryl- or cetylpyrimidinium chloride, imidazoline derivatives and compounds having cationic character, such as amine oxides, for example alkyldimethylamine oxides or alkylaminoethyldimethylamine oxides. In particular, the use of cetyltrimethylammonium salts is advantageous.

C. Amphoteric Surfactants

Amphoteric surfactants which can be used advantageously are

1. acyl/dialkylethylenediamine, for example sodium acyl amphoacetate, disodium acyl amphodipropionate, disodium alkyl amphodiacetate, sodium acyl amphohydroxypropylsulfonate, disodium acyl amphodiacetate and sodium acyl amphopropionate,

2. N-alkylamino acids, for example aminopropylalkylglutamide, alkylaminopropionic acid, sodium alkylimidodipropionate and lauroamphocarboxyglycinate.

D. Nonionic Surfactants

Nonionic surfactants which can be used advantageously are

1. alcohols,

2. alkanolamides, such as cocamides MEA/DEA/MIPA,

3. amine oxides, such as cocoamidopropylamine oxide,

4. esters which are formed by esterification of carboxylic acids with ethylene oxide, glycerol, sorbitan or other alcohols,

5. ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated propoxylated lanolin, ethoxylated/propoxylated polysiloxanes, propoxylated POE ethers and alkyl polyglycosides, such as lauryl glucoside, decyl glycoside and cocoglycoside,

6. sucrose esters, sucrose ethers

7. polyglycerol esters, diglycerol esters, monoglycerol esters

8. methylglucose esters, esters of hydroxy acids.

The cosmetic preparations according to the invention can comprise cosmetic auxiliaries as are customarily used in such preparations, e.g. preservatives, bactericides, antioxidants, perfumes, antifoams, dyes, pigments which have a coloring action, thickeners, surface-active substances, emulsifiers, emollient substances, moisturizing substances and/or humectant substances, fats, oils, waxes or other customary constituents of a cosmetic formulation, such as alcohols, polyols, polymers, foam stabilizers, electrolytes, pH-regulating substances, organic solvents or silicone derivatives.

According to the invention, it is also advantageous in some instances to add complexing agents to the preparations. Complexing agents are auxiliaries of cosmetology or of medicinal technology known per se. By complexing troublesome metals, such as Mn, Fe, Cu and others, it is possible, for example, to prevent undesired chemical reactions in cosmetic or dermatological preparations.

Complexing agents, in particular chelating agents, form complexes with metal atoms. In the presence of one or more polybasic complexing agents, i.e. chelating agents, these complexes are metallocycles. Chelates are compounds in which a single ligand occupies more than one coordination site on a central atom. In this case, normally extended compounds are thus closed as a result of complex formation via a metal atom or metal ion to form rings. The number of bonded ligands depends on the coordination number of the central metal. A prerequisite for the formation of the chelate is that the compound reacting with the metal contains two or more atomic groups which act as electron donors.

The complexing agent(s) can advantageously be chosen from the group of customary compounds, preferably at least one substance from the group consisting of tartaric acid and anions thereof, citric acid and anions thereof, aminopolycarboxylic acids and anions thereof (such as, for example, ethylenediaminetetraacetic acid (EDTA) and anions thereof, nitrilotriacetic acid (NTA) and anions thereof, hydroxyethylenediaminotriacetic acid (HOEDTA) and anions thereof, diethyleneaminopentaacetic acid (DPTA) and anions thereof, trans-1,2-diaminocyclohexanetetraacetic acid (CDTA) and anions thereof).

According to the invention, the complexing agent(s) is/are advantageously present in cosmetic or dermatological preparations preferably in amounts of from 0.01% by weight to 10% by weight, preferably from 0.05% by weight to 5% by weight, particularly preferably 0.1-2.0% by weight, based on the total weight of the preparations.

The examples below are intended to illustrate the subject matter of the invention in more detail without limiting it to them.

| | 1 | 2 | 3 |
|---|---|---|---|
| EXAMPLES | % by wt. | % by wt. | % by wt. |
| Decyl glucoside | 1.00 | 1.00 | — |
| Sodium lauroyl sarcosinate | — | — | 1.00 |
| Hydroxyethylcellulose | 0.25 | — | 0.25 |
| Xanthan gum | 0.35 | 0.20 | 0.35 |
| Acrylate/C₁₀₋₃₀-alkyl acrylate cross- | 0.35 | 0.25 | 0.35 |
| polymer | | | |
| PEG-40 hydrogenated castor oil | — | — | — |
| NaOH | 0.15 | 0.12 | 0.15 |
| Glycerol | 5.00 | 2.50 | 5.00 |
| Butylene glycol | — | 2.50 | — |
| Panthenol | 1.30 | 1.30 | 1.30 |
| Ethanol | 5.00 | — | 5.00 |
| Perfume, preservative, dyes | q.s. | q.s. | q.s. |
| Water | ad 100.00 | ad 100.00 | ad 100.00 |
| | 4 | 5 | 6 |
| EXAMPLES | % by wt. | % by wt. | % by wt. |
| Sodium lauroyl sarcosinate | 1.00 | 1.00 | 1.00 |
| Hydroxyethylcellulose | 0.20 | — | — |
| Xanthan gum | — | 0.20 | 0.35 |
| Acrylate/C₁₀₋₃₀-alkyl acrylate cross- | 0.30 | 0.20 | 0.25 |
| polymer | | | |
| PEG-40 hydrogenated castor oil | 1.00 | — | — |
| NaOH | 0.15 | 0.10 | 0.12 |
| Glycerol | 2.50 | 2.50 | 2.50 |
| Butylene glycol | 2.50 | 2.50 | 2.50 |
| Panthenol | 1.30 | 1.30 | 1.30 |
| Perfume, preservative, dyes | q.s. | q.s. | q.s. |
| Water | ad 100.00 | ad 100.00 | ad 100.00 |
| EXAMPLES | 7 | 8 | 9 |
| Product name | % by wt. | % by wt. | % by wt. |
| Sodium lauroyl sarcosinate | 1.00 | 1.00 | 1.00 |
| Xanthan gum | 0.35 | — | 0.30 |
| Acrylate/C₁₀₋₃₀-alkyl acrylate cross- | 0.25 | 0.32 | 0.20 |
| polymer | | | |
| PEG-40 hydrogenated castor oil | 0.50 | 1.00 | 0.50 |
| NaOH | 0.12 | 0.16 | 0.12 |
| Glycerol | 2.50 | 2.50 | 5.00 |
| Butylene glycol | 2.50 | 2.50 | — |
| Panthenol | 1.30 | 1.30 | 1.30 |
| Perfume, preservative, dyes | q.s. | q.s. | q.s. |
| Water | ad 100.00 | ad 100.00 | ad 100.00 |

The preparations according to examples 1-9 are combined manner at 25° C. and stirred until a homogeneous mass is formed.

That which is claimed:

1. A cosmetic preparation, comprising:
(a) one or more N-acyl sarcosinates;
(b) one or more polysaccharides;
(c) one or more substances that form a gel structure in water, that are not polysaccharides and that have interface-active properties;
(d) one or more solubility promoters; and
(e) water.

2. The preparation as claimed in claim 1, wherein the one or more N-acyl sarcosinates include sodium lauryl sarcosinate.

3. The preparation as claimed in claim 1, wherein the one or more N-acyl sarcosinates are present in an amount from 0.01 to 10% by weight, based on the total weight of the preparation.

4. The preparation as claimed in claim 1, wherein the one or more N-acyl sarcosinates are present in an amount from 0.01 to 5% by weight, based on the total weight of the preparation.

5. The preparation as claimed in claim 1, wherein the one or more N-acyl sarcosinates are present in an amount from 0.5 to 3% by weight, based on the total weight of the preparation.

6. The preparation as claimed in claim 1, wherein the one or more polysaccharides include xanthan gum.

7. The preparation as claimed in claim 1, wherein the one or more polysaccharides are present in an amount from 0.01 to 10% by weight, based on the total weight of the preparation.

8. The preparation as claimed in claim 1, wherein the one or more polysaccharides are present in an amount from 0.01 to 5% by weight, based on the total weight of the preparation.

9. The preparation as claimed in claim 1, wherein the one or more polysaccharides are present in an amount from 0.5 to 3% by weight, based on the total weight of the preparation.

10. The preparation as claimed in claim 1, wherein the one or more substances that form a gel structure in water include Carbomer 1342.

11. The preparation as claimed in claim 1, wherein the one or more substances that form a gel structure in water are present in an amount from 0.01 to 10% by weight, based on the total weight of the preparation.

12. The preparation as claimed in claim 1, wherein the one or more substances that form a gel structure in water are present in an amount from 0.01 to 5% by weight, based on the total weight of the preparation.

13. The preparation as claimed in claim 1, wherein the one or more substances that form a gel structure in water are present in an amount from 0.5 to 3% by weight, based on the total weight of the preparation.

14. The preparation as claimed in claim 1, wherein the one or more solubility promoters include solubility promoters selected from the group consisting of polyethoxylated saturated fat or oil components.

15. The preparation as claimed in claim 1, wherein the one or more solubility promoters include PEG-40 hydrogenated castor oil.

16. The preparation as claimed in claim 1, wherein the one or more solubility promoters are present in an amount from 0.01 to 10% by weight, based on the total weight of the preparation.

17. The preparation as claimed in claim 1, wherein the one or more solubility promoters are present in an amount from 0.01 to 5% by weight, based on the total weight of the preparation.

18. The preparation as claimed in claim 1, wherein the one or more solubility promoters are present in an amount from 0.5 to 3% by weight, based on the total weight of the preparation.

19. A shaving gel, which is suitable for use with a mechanical shaving device, comprising:
(a) from 0.01 to 10% by weight, based on the total weight of the preparation, of one or more N-acyl sarcosinates;
(b) from 0.01 to 10% by weight, based on the total weight of the preparation, of one or more polysaccharides;
(c) from 0.01 to 10% by weight, based on the total weight of the preparation, of one or more substances that form a gel structure in water, that are not polysaccharides and that have interface-active properties;
(d) from 0.01 to 10% by weight, based on the total weight of the preparation, of one or more solubility promoters including solubility promoters selected from the group consisting of polyethoxylated saturated fat or oil components; and
(e) water.

20. A method of shaving using a mechanical shaving device, comprising the steps of:
applying a shaving gel to the skin surface, the shaving gel comprising:
(a) one or more N-acyl sarcosinates;
(b) one or more polysaccharides;
(c) one or more substances that form a gel structure in water, that are not polysaccharides and that have interface-active properties;
(d) one or more solubility promoters; and
(e) water; and
shaving along the skin surface using a mechanical shaving device.
